(19) United States
(12) Patent Application Publication
(10) Pub. No.: US 2010/0101580 A1
(43) Pub. Date: Apr. 29, 2010

(54) SECUREMENT DEVICE FOR CONTINUOUS POSITIVE AIRWAY PRESSURE APPARATUS

(76) Inventors: Katherine Stumm, Afton, TN (US); Lily Lam, Afton, TN (US)

Correspondence Address: LAW OFFICE OF JERRY D. HAYNES , P.A, P.O. Box 350392, Fort Lauderdale, FL, 33335, US

(21) Appl. No.: 12/258,481

(22) Filed: Oct. 27, 2008

(51) Int. Cl. A61M 16/06

(52) U.S. Cl. 128/205.25

(57) ABSTRACT

Disclosed is a securement device for securing a nasal pillow of a continuous positive airway pressure (CPAP) apparatus to a user. The securement device includes an adjustable elongated band and a set of grommets. The adjustable elongated band has a first end portion and a second end portion. The adjustable elongated band is adapted to assume a looped position by disposing the first end portion and the second end portion adjacent to each other. Each of the first end portion and the second end portion includes an aperture configured therein. The set of grommets is disposed on the aperture in the first end portion and the second end portion. The set of grommets is adapted to secure the nasal pillow between the first end portion and the second end portion of the adjustable elongated band in the looped position thereof.

### FIELD OF THE INVENTION

The present invention generally relates to securement devices, and, more particularly, to a securement device for securing a nasal pillow of a continuous positive airway pressure (CPAP) apparatus to a user.

### BACKGROUND OF THE INVENTION

Sleep apnea is a sleep disorder characterized by pauses in breathing during sleep. Sleep apnea reduces oxygen in the blood supply and causes arousal from sleep. Generally, a continuous positive airway pressure (CPAP) apparatus is used by a user for artificial ventilation that prevents sleep apnea by delivering a stream of compressed air into the respiratory system of the user. The CPAP apparatus is configured to supply air at a prescribed pressure, usually determined by a physician, into the respiratory system of the user.

The CPAP apparatus includes a flow generator, a hose, a nasal pillow and a headband. The flow generator is configured to generate and supply compressed air. The hose is configured to connect the flow generator to the nasal pillow. The nasal pillow is disposed on a nose of a user and is configured to deliver a stream of compressed air into the respiratory system of the user. The nasal pillow is secured to the head portion of the user by means of the headband.

There are a variety of existing headbands that are preferably used for securing the nasal pillow to the head portion of the user. However, these headbands have numerous limitations. For example, these headbands are prone to slippage while being used, thereby causing slippage of the nasal pillow. Further, such headbands exert undue pressure on the head of a user. Furthermore, such headbands are prone to entangle with the hair of the user, thereby being cumbersome and uncomfortable.

Accordingly, based on the foregoing, there exists a need for a securement device for securing the nasal pillow of the CPAP apparatus to the head portion of the user, which is capable of preventing slippage of the nasal pillow during use. Further, there is a need for a securement device which avoids entanglement with the hair of the user. Still further, there is a need for a securement device, which is simple in construction, comfortable to wear and easy to use.

### SUMMARY OF THE INVENTION

In view of the foregoing disadvantages inherent in the prior art, the general purpose of the present invention is to provide a securement device for a nasal pillow of a CPAP apparatus that is configured to include all the advantages of the prior art, and to overcome the drawbacks inherent therein.

Accordingly, an object of the present invention is to provide a securement device for a nasal pillow of a CPAP apparatus, which is capable of preventing slippage of the nasal pillow during use.

Another object of the present invention is to provide a securement device for a nasal pillow of a CPAP apparatus, which avoids entanglement with the hair of a user.

Yet another object of the present invention is to provide a securement device for a nasal pillow of a CPAP apparatus, which is simple in construction, comfortable to wear and easy to use.

In light of the above objects, a securement device for securing a nasal pillow of a continuous positive airway pressure (CPAP) apparatus to a user is disclosed. The securement device includes an adjustable elongated band and a set of grommets. The adjustable elongated band has a first end portion and a second end portion. The adjustable elongated band is adapted to assume a looped position by disposing the first end portion and the second end portion adjacent to each other. Each of the first end portion and the second end portion includes an aperture configured therein. The set of grommets is disposed on the aperture in the first end portion and the second end portion. The set of grommets is adapted to secure the nasal pillow between the first end portion and the second end portion of the adjustable elongated band in the looped position thereof. Upon securing the nasal pillow between the first end portion and the second end portion in the looped position of the adjustable elongated band, the securement device is adapted to be received around a head portion of the user, thereby securing the nasal pillow to the user.

In one aspect of the present invention, the adjustable elongated band is composed of a neoprene material.

In another aspect of the present invention, the adjustable elongated band is composed of an elastic terry cloth material.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, in which:

FIG. 1A illustrates an exploded view of a securement device for a nasal pillow of a CPAP apparatus, in accordance with an embodiment of the present invention;

FIG. 1B illustrates a perspective view depicting a looped position of an adjustable elongated band of the securement device shown in the FIG. 1A, in accordance with an embodiment of the present invention; and

FIG. 2 illustrates a view of the securement device of FIG. 1A securing a nasal pillow of a CPAP apparatus to a head portion of a user, in accordance with an embodiment of the present invention.

Like reference numerals refer to like parts throughout the description of several views of the drawings.

### DETAILED DESCRIPTION OF THE INVENTION

The exemplary embodiments described herein detail for illustrative purposes are subject to many variations in structure and design. It should be emphasized, however, that the present invention is not limited to a particular securement device for a nasal pillow of a CPAP apparatus as shown and described. It is understood that various omissions and substitutions of equivalents are contemplated as circumstances may suggest or render expedient, but these are intended to cover the application or implementation without departing from the spirit or scope of the claims of the present invention.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

The present invention provides a securement device for securing a nasal pillow of a CPAP apparatus to a head portion of a user. The securement device is adapted to be adjustable in length for being utilized by users of different age groups.

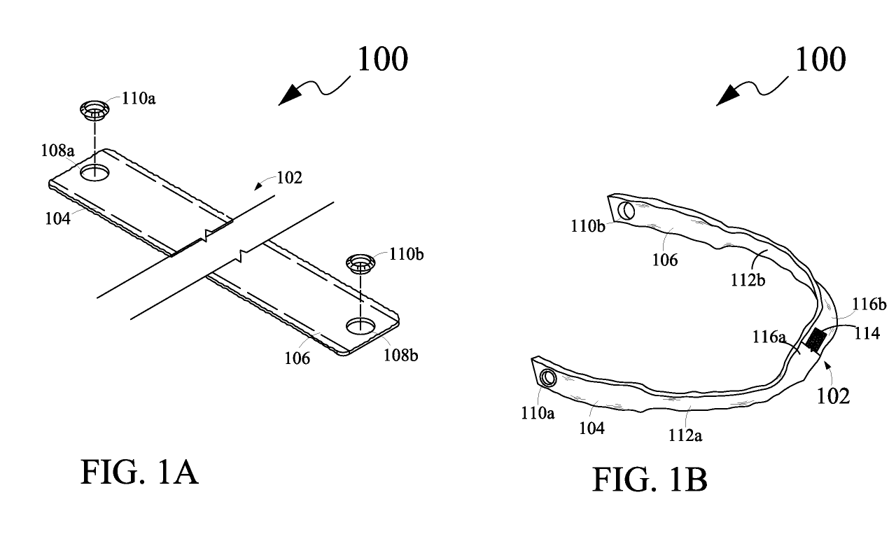

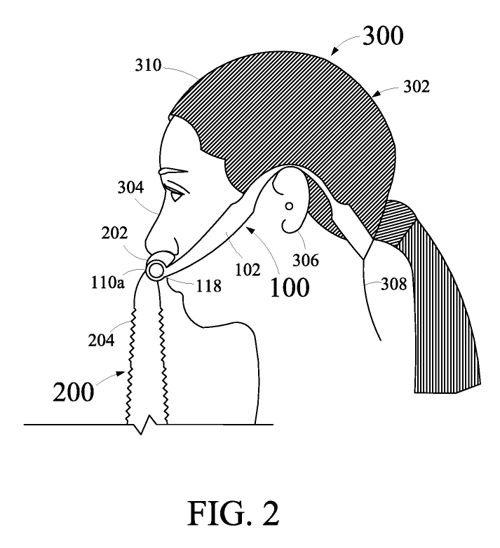

Referring to FIGS. 1A, 1B and 2, a securement device 100 adapted to be utilized with a CPAP apparatus 200 is illustrated. More specifically, FIG. 1A illustrates an exploded view of the securement device 100, in accordance with an embodiment of the present invention, FIG. 1B illustrates a perspective view depicting a looped position of an adjustable elongated band of the securement device 100 shown in the FIG. 1A, in accordance with an embodiment of the present invention, and FIG. 2 illustrates a view of the securement device 100 securing a nasal pillow 202 of the CPAP apparatus 200 to a user 300, in accordance with an embodiment of the present invention. The CPAP apparatus 200 (shown in FIG. 2), as disclosed herein, includes a flow generator (not shown), the nasal pillow 202 and a hose 204 coupled thereto. The flow generator is configured to generate and supply compressed air. The hose 204 is configured to transport the compressed air from the flow generator to the nasal pillow 202. The nasal pillow 202 is received on a nose 304 of the user 300. The nasal pillow 202 is configured to deliver the stream of compressed air into a respiratory system of the user 300.

As disclosed herein, the securement device 100 includes an adjustable elongated band 102 as shown in FIG. 1A and FIG. 1B. The adjustable elongated band 102 includes a first end portion 104 and a second end portion 106. The first end portion 104 and the second end portion 106 include an aperture 108a and an aperture 108b, respectively, configured therein. The aperture 108a and the aperture 108b may be collectively referred to as “apertures 108”.

The apertures 108 are adapted to receive a set of grommets thereon. More specifically, a grommet 110a is disposed on the aperture 108a and a grommet 110b is disposed on the aperture 108b. The grommet 110a and the grommet 110b are hereinafter collectively referred to as “grommets 110”. The grommets 110 may be composed from various materials, such as metal, plastic, wood and the like. The grommets 110 are configured to prevent ripping of the apertures 108.

Further, as shown in FIG. 1B, the adjustable elongated band 102 includes a pair of strips, such as a strip 112a and a strip 112b (hereinafter collectively referred to as “pair of strips 112” ). Specifically, the pair of strips 112 is adapted to be removably secured to each other by fastening means 114 configured at end portions of the pair of strips 112 for configuring the adjustable elongated band 102. For example, a portion of the fastening means 114 is configured at an end portion 116a of the strip 112a and a corresponding portion of the fastening means 114 is configured at an end portion 116b of the strip 112b. In one embodiment of the present invention, the fastening means 114 may be a hook and loop fastener, such that the hook fastener is configured at one of the end portions 116a and 116b of the pair of strips 112 and the loop fastener is configured at the other of the end portions 116a and 116b. The strip 112a may be removably secured to the strip 112b by means of the fastening means 114 by securing the hook fastener to the loop fastener. Further, the fastening means 114 may enable a user to change a length of the elongated band 102 as per his/her requirement by varying an overlapping length of the hook fastener with the loop fastener. Further, it will be evident to a person skilled in the art that the fastening means 114, as described herein, is for purpose of explanation only and must not be construed as limiting to the scope of the present invention.

Further, the adjustable elongated band 102 is adapted to assume a looped position (shown in FIG. 1B) by disposing the first end portion 104 and the second end portion 106 adjacent to each other. In the looped position of the adjustable elongated band 102, the grommets 110 are adapted to secure the nasal pillow 202 (shown in FIG. 2) between the first end portion 104 and the second end portion 106. More specifically, the grommets 110 are adapted to receive at least one attachment means 118 configured on the nasal pillow 202 for securing the nasal pillow 202 to the adjustable elongated band 102, when the adjustable elongated band 102 assumes the looped position. In one embodiment of the present invention, the least one attachment means 118 may include a pair of attachment bands coupled to the nasal pillow 202 with the help of quick release clips. The attachment bands may be received through the grommets 110 and tied thereto for securing the nasal pillow 202 to the adjustable elongated band 102. In another embodiment of the present invention, the least one attachment means 118 may include elongated projecting members configured on the nasal pillow 202. The elongated projecting members may be received in the grommets 110 and secured thereto by means of a nut, a clamp and the like. Upon receiving the nasal pillow 202 to the adjustable elongated band 102, the adjustable elongated band 102 may be positioned on a head portion 302 of the user 300 for securing the nasal pillow 202 to the user 300.

More specifically, in use, the user 300 may enable the looped position of the adjustable elongated band 102 by disposing the first end portion 104 and the second end portion 106 adjacent to each other, as shown in FIG. 1B. Thereafter, the grommets 110 disposed on the first end portion 104 and the second end portion 106 of the adjustable elongated band 102 may receive the at least one attachment means 118 coupled to the nasal pillow 202, thereby securing the nasal pillow 202 to the adjustable elongated band 102. Subsequently, the user 300 may dispose the nasal pillow 202 on the nose 304, such that the adjustable elongated band 102 extends from the face portion of the user 300 to be supported above the ears, such as an ear 306, of the user 300 and positioned around a nape portion 308, as shown in FIG. 2. Accordingly, the securement device 100 securely positions the nasal pillow 202 on the head portion 302 of the user 300, thereby preventing any slippage of the nasal pillow 202 during use. In one embodiment of the present invention the adjustable elongated band 102 may be positioned below a hairline of the user 300. Accordingly, the adjustable elongated band 102 is adapted to have minimal contact with hair 310 of the user 300, thereby avoiding entanglement with the hair 310.

The securement device 100, as disclosed herein, is subject to many variations. In one embodiment of the present invention, the adjustable elongated band 102 may be composed from various soft and elastic materials such as neoprene. In another embodiment of the present invention, the adjustable elongated band 102 may be composed of an elastic terry cloth material. Further, in one embodiment of the present invention, an inner surface (not shown) of the adjustable elongated band 102, which is in contact with a skin of the user 300, may be disposed with a felt strip or a rubberized strip for enabling comfortable contact between the skin of the user and the inner surface of the adjustable elongated band 102 for preventing slippage of the securement device 100 and the nasal pillow 202.

Various embodiments of the present invention offer following advantages. The securement device, as described herein, is capable of securing a nasal pillow of a CPAP apparatus to a head portion of a user and prevents slippage of the nasal pillow from the head portion during use. More specifically, the securement device remains stable in any sleeping position of the user, thereby ensuring a sound sleep to the user without any interruption. Further, the securement device, while being used, does not entangle with the hair of the user and accordingly the securement device may be conveniently and comfortably used for securing the nasal pillow to the user. Furthermore, the securement device is simple in construction, comfortable to wear and easy to use.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, and to thereby enable others skilled in the art to best utilize the present invention and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the spirit or scope of the claims of the present invention.

What is claimed is:

1. A securement device for securing a nasal pillow of a continuous positive airway pressure (CPAP) apparatus to a user, the securement device comprising:
an adjustable elongated band having a first end portion and a second end portion, each of the first end portion and the second end portion comprising an aperture configured therein, wherein the adjustable elongated band is adapted to assume a looped position by disposing the first end portion and the second end portion adjacent to each other; and
a set of grommets disposed on the aperture in the first end portion and the second end portion, the set of grommets adapted to secure the nasal pillow between the first end portion and the second end portion of the adjustable elongated band in the looped position thereof,
wherein upon securing the nasal pillow between the first end portion and the second end portion in the looped position of the adjustable elongated band, the securement device is adapted to be received around a head portion of the user, thereby securing the nasal pillow to the user.

2. The securement device of claim 1, wherein the adjustable elongated band is composed of a neoprene material.

3. The securement device of claim 1, wherein the adjustable elongated band is composed of an elastic terry cloth material.

4. The securement device of claim 1, wherein the adjustable elongated band is composed of an elastic material.

5. The securement device of claim 1, wherein the set of grommets is composed of a plastic material.

6. The securement device of claim 1, wherein the adjustable elongated band comprises a pair of strips adapted to be removably secured to each other by means of a fastening means disposed at corresponding end portions of the pair of strips.

7. The securement device of claim 6, wherein the fastening means comprises a hook and loop fastener.
